(19) United States
(12) Patent Application Publication
(10) Pub. No.: US 2026/0051077 A1
(43) Pub. Date: Feb. 19, 2026

(54) SYSTEM AND METHOD FOR SITUATIONAL AWARENESS IN CARDIAC CATHERIZATION

(71) Applicant: GE Precision Healthcare LLC, Waukesha, WI (US)

(72) Inventor: Hani Nozari MIRAR, Oslo (NO)

(21) Appl. No.: 18/808,576

(22) Filed: Aug. 19, 2024

(51) Int. Cl. G06T 7/70; G06T 7/11

(52) U.S. Cl. CPC G06T 7/70; G06T 7/11; G06T 2207/10136

(57) ABSTRACT

Various systems and methods are provided for displaying information indicating whether a distance, between a position of a feature in a region of interest of a subject and a position of a medical instrument in the region of interest, is within a safety range corresponding to the feature. The position of the feature in the region of interest may be determined. The position of the medical instrument in the region of interest may be determined. The distance between the position of the feature and the position of the medical instrument may be determined. Whether the distance is within the safety range may be determined. A display may be controlled to display an ultrasound image of the region of interest including the feature and the medical instrument, and display the information indicating whether the distance is within the safety range.

### TECHNICAL FIELD

The present disclosure relates to a system and method for displaying information indicating whether a distance between a position of a feature of a region of interest of a subject and a position of a medical instrument in the region of interest is within a safety range corresponding to the feature.

### BACKGROUND

During an interventional procedure, a clinician might navigate a medical instrument through a region of interest of a subject. For example, during a cardiac procedure, a clinician may navigate a catheter through a heart of a subject in order to deliver a stent, ablate tissue, remove a thrombus, analyze cardiac function, or the like. The region of interest may include various features that the medical instrument should not contact, or come into close contact with, in order to maintain safety of the subject. For example, in thermal ablation implementations, the close proximity of an ablation catheter to a permanent pacing device might increase the risk of pacing dysfunctions for both pacemakers and defibrillators. As another example, the His bundle, which travels through the membranous septum in immediate proximity to the posterior sinus of the Valsalva and runs just under the left ventricular endocardium, is anatomically vulnerable to mechanical trauma during catheterization. A single touch of these structures by a catheter tip may cause intra-His bundle injury resulting in complete heart blockage. As yet another example, manipulation of a catheter within the heart may inadvertently dislodge thrombi. As yet another example, utilizing an improper technique or aggressively manipulating a cardiac catheter may result in injuries to the internal walls or valves of the heart. As such, the clinician should be cognizant of the positon of the medical instrument in the region of interest and the relative distances between the medical instrument and various features of the region of interest. Further, the clinician should be cognizant of how closely the medical instrument can be safely positioned to these various features to maintain subject safety.

### SUMMARY

This summary introduces concepts that are described in more detail in the detailed description. It should not be used to identify essential features of the claimed subject matter, nor to limit the scope of the claimed subject matter.

In an aspect, a system may include an ultrasound probe configured to acquire ultrasound data of a region of interest of a subject; a display configured to display an ultrasound image corresponding to the ultrasound data; a memory configured to store instructions; and one or more processors configured to execute the instructions to: receive information identifying a feature in the region of interest of the subject to be tracked during an interventional procedure involving a medical instrument that is navigated through the region of interest; determine a position of the feature in the region of interest of the subject; determine a position of the medical instrument in the region of interest of the subject during the interventional procedure; determine a distance between the position of the feature and the position of the medical instrument; determine whether the distance is within a safety range corresponding to the feature; control the display to display the ultrasound image of the region of interest including the feature and the medical instrument; and control the display to display information indicating whether the distance between the position of the feature and the position of the medical instrument is within the safety range corresponding to the feature.

In another aspect, a method may include receiving information identifying a feature in the region of interest of the subject to be tracked during an interventional procedure involving a medical instrument that is navigated through the region of interest; determining a position of the feature in the region of interest of the subject; determining a position of the medical instrument in the region of interest of the subject during the interventional procedure; determining a distance between the position of the feature and the position of the medical instrument; determining whether the distance is within a safety range corresponding to the feature; controlling the display to display the ultrasound image of the region of interest including the feature and the medical instrument; and controlling the display to display information indicating whether the distance between the position of the feature and the position of the medical instrument is within the safety range corresponding to the feature.

In yet another aspect, a non-transitory computer-readable medium may store instructions that, when executed by one or more processors, cause the one or more processors to: receive information identifying a feature in the region of interest of the subject to be tracked during an interventional procedure involving a medical instrument that is navigated through the region of interest; determine a position of the feature in the region of interest of the subject; determine a position of the medical instrument in the region of interest of the subject during the interventional procedure; determine a distance between the position of the feature and the position of the medical instrument; determine whether the distance is within a safety range corresponding to the feature; control a display to display an ultrasound image of the region of interest including the feature and the medical instrument; and control the display to display information indicating whether the distance between the position of the feature and the position of the medical instrument is within the safety range corresponding to the feature.

### BRIEF DESCRIPTION OF DRAWINGS

FIG. 1 is a diagram of an example system for displaying information indicating whether a distance between a position of a feature of a region of interest of a subject and a position of a medical instrument in the region of interest is within a safety range corresponding to the feature.

FIG. 2 is a diagram of an example ultrasound system for acquiring ultrasound data of a region of interest of a subject, and displaying information indicating whether a distance between a position of a feature of a region of interest of a subject and a position of a medical instrument in the region of interest is within a safety range corresponding to the feature.

FIG. 3 is a diagram of an example tracking system for acquiring tracking data of a medical instrument located within a region of interest of a subject.

FIG. 4 is a diagram of an example preoperative imaging system for acquiring preoperative imaging data of a region of interest of a subject.

FIG. 5 is a flowchart of an example process for displaying information indicating whether a distance between a position of a feature of a region of interest of a subject and a position of a medical instrument in the region of interest is within a safety range corresponding to the feature.

FIG. 6 is a diagram of an ultrasound image displaying features of a region of interest, a medical instrument, safety ranges of the features, and information indicating whether respective positions of the features are within the safety ranges of the features.

FIG. 7 is a diagram of an ultrasound image displaying features of a region of interest and a medical instrument.

FIG. 8 is a diagram of an ultrasound image displaying features of a region of interest, a medical instrument, and information indicating whether respective positions of the features are within the safety ranges of the features.

FIG. 9 is a diagram of an ultrasound image displaying features of a region of interest, a medical instrument, and information indicating whether respective positions of the features are within the safety ranges of the features.

FIG. 10 is a diagram of an ultrasound image displaying features of a region of interest, a medical instrument, and information indicating whether respective positions of the features are within the safety ranges of the features.

FIG. 11 is a diagram of a map displaying features of a region of interest, a medical instrument, and information indicating whether respective positions of the features are within the safety ranges of the features.

### DETAILED DESCRIPTION

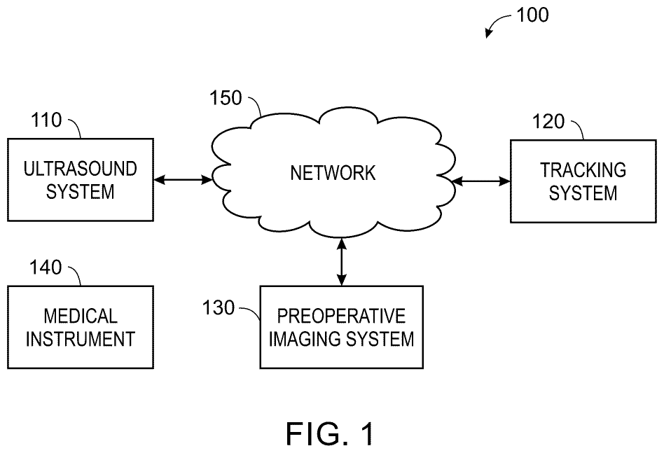

FIG. 1 is a diagram of an example system 100 for displaying information indicating whether a distance between a position of a feature of a region of interest of a subject and a position of a medical instrument in the region of interest is within a safety range corresponding to the feature. As shown in FIG. 1, the system 100 may include an ultrasound system 110, a tracking system 120, a preoperative imaging system 130, a medical instrument 140, and a network 150.

The ultrasound system 110 may be configured to acquire ultrasound data of a region of interest of a subject. For example, the ultrasound system 130 may be a two-dimensional (2D) ultrasound system, a three-dimensional (3D) ultrasound system, a four-dimensional (4D) ultrasound system, a Doppler ultrasound system, or the like. The subject may be a person, an animal, a phantom, or the like. The region of interest may be any anatomical region of the subject. For example, the region of interest may be a heart, a brain, an organ, a blood vessel, or the like.

The tracking system 120 may be configured to acquire tracking data of the medical instrument 140 located within the region of interest of the subject. For example, the tracking system 120 may be an electromagnetic tracking system, an optical tracking system, an acoustic tracking system, an inertial tracking system, an ultrasound tracking system, or the like.

The preoperative imaging system 130 may be configured to acquire preoperative imaging data of the region of interest of the subject. For example, the preoperative imaging system 130 may be a computed tomography (CT) system, a magnetic resonance imaging (MRI) system, an ultrasound system, an X-ray system, a positron emission tomography (PET) device, or the like.

The medical instrument 140 may be any medical instrument that can be navigated through a region of interest of a subject. For example, the medical instrument 140 may be a catheter, a needle, a trocar, a cannula, or the like. The medical instrument 140 may be used for various interventional procedures involving the region of interest. For example, a catheter may be used for delivering a stent to an occluded blood vessel, ablating tissue, analyzing cardiac function, removing a thrombus from an occluded blood vessel, or the like. Alternatively, the medical instrument 140 may be an implantable device that is to be implanted in the heart of the subject. For example, the implantable device may be a pacemaker, a stent, a defibrillator, a left ventricular assist device, a valve clip, or the like. Alternatively, the medical instrument 140 may be any object that can be navigated throughout the region of interest and/or tracked through the region of interest.

The network 150 may permit communication between the ultrasound system 110, the tracking system 120, and the preoperative imaging system 130. For example, the network 150 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The number and arrangement of the system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

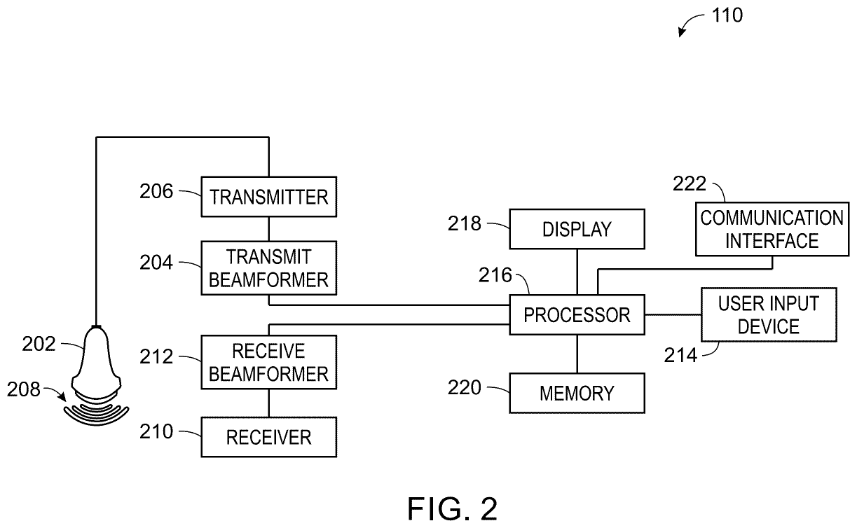

FIG. 2 is a diagram of example components of the ultrasound system 110 for acquiring ultrasound data of a region of interest of a subject, and displaying information indicating whether a distance between a position of a feature of a region of interest of a subject and a position of a medical instrument in the region of interest is within a safety range corresponding to the feature. As shown in FIG. 2, the ultrasound system 110 may include an ultrasound probe 202, a transmit beamformer 204, a transmitter 206, elements 208 a receiver 210, a receive beamformer 212, a user input device 214, a processor 216, a display 218, a memory 220, and a communication interface 222. The foregoing components may be connected via wired or wireless connections.

The ultrasound probe 202 may be configured to acquire ultrasound data. For example, the ultrasound probe 202 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 202 may be configured to generate ultrasound signals, emit the ultrasound signals towards the region of interest of a subject, receive echo ultrasound signals that are back-scattered from the region of interest of the subject, generate ultrasound data based on the echo ultrasound signals, and output the ultrasound data.

The transmit beamformer 204 may be configured to apply delay times to electrical signals provided to the elements 208 to focus corresponding ultrasound signals at the region of interest. The transmitter 206 may be configured to transmit electrical signals to the elements 208 to drive the elements 208 to emit ultrasound signals towards the region of interest. The elements 208 may be configured to receive the electrical signals from the transmitter 206, convert the electrical signals into ultrasound signals, and emit the ultrasound signals towards the region of interest. The elements 208 may be configured to receive echo ultrasound signals that are back-scattered by the region of interest, convert the echo ultrasound signals into electrical signals, and provide the electrical signals to the receiver 210. The receiver 210 may be configured to receive electrical signals from the elements 208, and provide the electrical signals to the receive beamformer 212. The receive beamformer 212 may apply delay times to the electrical signals received from the elements 208.

The user input device 214 may be configured to receive a user input, and provide the user input to the processor 216. For example, the user input device 214 may be a touch screen display, a keyboard, a keypad, a mouse, a button, a switch, a microphone, or the like. Additionally, or alternatively, the user input device 214 may be configured to sense information. For example, the user input device 214 may sense information from an electro-magnetic positioning system, an inertial measurement system, an accelerometer, a gyroscope, an actuator, or the like.

The processor 216 may be configured to perform the operations as described herein. For example, the processor 216 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. The processor 216 may be implemented in hardware, firmware, or a combination of hardware and software. The processor 216 may include one or more processors 216 configured to perform the operations described herein. For example, a single processor 216 may be configured to perform all of the operations described herein. Alternatively, multiple processors 216, collectively, may be configured to perform all of the operations described herein, and each of the multiple processors 216 may be configured to perform a subset of the operations descried herein. For example, a first processor 216 may perform a first subset of the operations described herein, a second processor 216 may be configured to perform a second subset of the operations described herein, etc.

The processor 216 may be configured to control the ultrasound probe 202 to acquire ultrasound data. The processor 216 may be configured to control which of the elements 208 are active, and control the shape of a beam emitted from the ultrasound probe 202. The processor 216 may generate ultrasound images for display. For example, the processor 216 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 218 may be configured to display information. For example, the display 218 may be a monitor, an LED display, a cathode ray tube, a projector display, a touchscreen, tablet computer, mobile phone, or the like. The display 218 may display ultrasound images based on the ultrasound data in real-time. For example, the display 218 may display the ultrasound images within one second, two seconds, five seconds, etc., of the ultrasound data being acquired by the ultrasound probe 202.

The memory 220 may be configured to store information and/or instructions for use by the processor 216. The memory 220 may be a non-transitory computer-readable medium. For example, the memory 220 may be a random access memory (RAM), a read only memory (ROM), and/or another type of dynamic or static storage device (e.g., a flash memory, a magnetic memory, and/or an optical memory) that stores information and/or instructions for use by the processor 216. The memory 220 may be configured to store instructions that, when executed by the processor 216, cause the processor 216 to perform the operations described herein.

The communication interface 222 may be configured to enable the processor 216 to communicate with other systems, such as via a wired connection, a wireless connection, or a combination of wired and wireless connections. For example, the communication interface 222 may include an Ethernet interface, an optical interface, a coaxial interface, an infrared interface, a radio frequency (RF) interface, a universal serial bus (USB) interface, a Wi-Fi interface, a cellular network interface, or the like.

The number and arrangement of the components of the ultrasound system 110 shown in FIG. 2 are provided as an example. In practice, the ultrasound system 110 may include additional components, fewer components, different components, or differently arranged components than those shown in FIG. 2. Additionally, or alternatively, a set of components (e.g., one or more components) of the ultrasound system 110 may perform one or more functions described as being performed by another set of components of the ultrasound system 110.

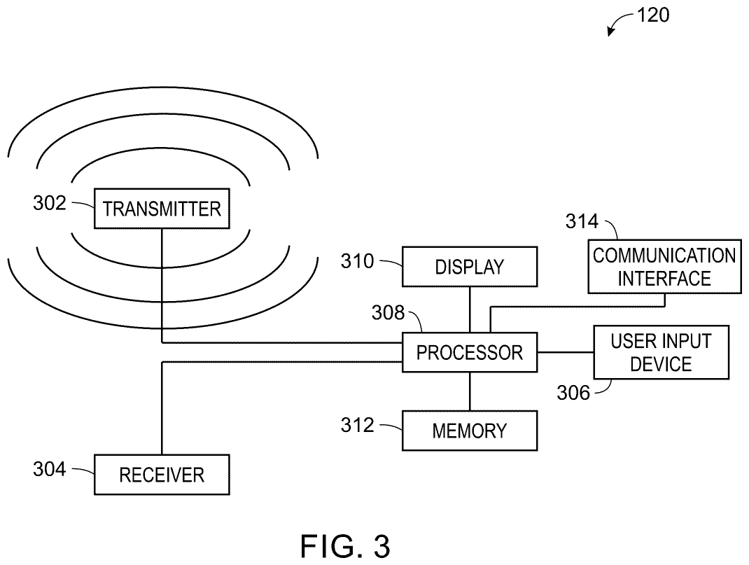

FIG. 3 is a diagram of example components of a tracking system 120. As shown in FIG. 3, the tracking system 120 may include a transmitter 302, a receiver 304, a user input device 306, a processor 308, a display 310, a memory 312, and a communication interface 314.

The transmitter 302 may be configured to generate a magnetic field. The receiver 304 may be configured to output a signal in response to the magnetic field generated by the transmitter 302. The processor 308 may receive the output signal from the receiver 304, and acquire tracking data that identifies a position and/or an orientation of the receiver 304. According to an embodiment, the receiver 304 may be attached to the ultrasound probe 202 to track a position and/or an orientation of the ultrasound probe 202. Alternatively, the receiver 304 may be attached to the medical instrument 140 to track a position and/or an orientation of the medical instrument 140. Alternatively, the receiver 304 may be attached to the feature in the region of interest.

The user input device 306 may be configured to receive a user input, and provide the user input to the processor 308. For example, the user input device 306 may be a touch screen display, a keyboard, a keypad, a mouse, a button, a switch, a microphone, or the like. Additionally, or alternatively, the user input device 306 may be configured to sense information. For example, the user input device 306 may sense information from an electro-magnetic positioning system, an inertial measurement system, an accelerometer, a gyroscope, an actuator, or the like.

The processor 308 may be configured to perform the operations as described herein. For example, the processor 308 may be a CPU, a GPU, an APU, a microprocessor, a microcontroller, a DSP, an FPGA, an ASIC, or the like. The processor 308 may be implemented in hardware, firmware, or a combination of hardware and software. The processor 308 may include one or more processors 308 configured to perform the operations described herein. For example, a single processor 308 may be configured to perform all of the operations described herein. Alternatively, multiple processors 308, collectively, may be configured to perform all of the operations described herein, and each of the multiple processors 308 may be configured to perform a subset of the operations descried herein. For example, a first processor 308 may perform a first subset of the operations described herein, a second processor 308 may be configured to perform a second subset of the operations described herein, etc.

The processor 308 may be configured to control the transmitter 308 to acquire tracking data. The processor 308 may be configured to control excitations of the transmitter 302 to generate a magnetic field. The processor 308 may acquire tracking data based on controlling the transmitter 302.

The display 310 may be configured to display information. For example, the display 310 may be a monitor, an LED display, a cathode ray tube, a projector display, a touchscreen, tablet computer, mobile phone, or the like. The display 310 may display the tracking data in real-time. For example, the display 310 may display the tracking data within one second, two seconds, five seconds, etc., of the tracking data being acquired.

The memory 312 may be configured to store information and/or instructions for use by the processor 308. The memory 312 may be a non-transitory computer-readable medium. For example, the memory 312 may be a RAM, a ROM, a flash memory, a magnetic memory, an optical memory, or the like. The memory 312 may be configured to store instructions that, when executed by the processor 308, cause the processor 308 to perform the operations described herein.

The communication interface 314 may be configured to enable the processor 308 to communicate with other systems, such as via a wired connection, a wireless connection, or a combination of wired and wireless connections. For example, the communication interface 314 may include an Ethernet interface, an optical interface, a coaxial interface, an infrared interface, an RF interface, a USB interface, a Wi-Fi interface, a cellular network interface, or the like.

The number and arrangement of the components of the tracking system 120 shown in FIG. 3 are provided as an example. In practice, the tracking system 120 may include additional components, fewer components, different components, or differently arranged components than those shown in FIG. 3. Additionally, or alternatively, a set of components (e.g., one or more components) of the tracking system 120 may perform one or more functions described as being performed by another set of components of the tracking system 120.

Although FIG. 3 depicts the tracking system 120 as being an electromagnetic tracking system, it should be understood that the embodiments herein are applicable to other types of tracking systems, such as optical tracking systems, acoustic tracking systems, ultrasound tracking systems, AI-based tracking methods, or the like.

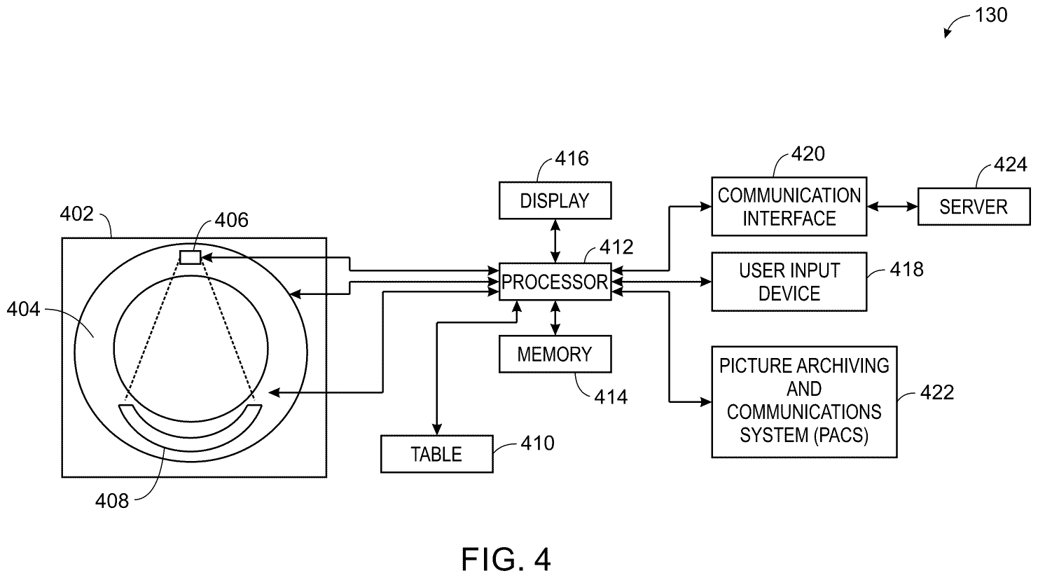

FIG. 4 is a diagram of an example preoperative imaging system 130 for acquiring preoperative imaging data of a region of interest of a subject. As shown in FIG. 4, the preoperative imaging system 130 may include a gantry 402, a rotational frame 404, an X-ray source 406, an X-ray detector 408, a table 410, a processor 412, a memory 414, a display 416, a user input device 418, a communication interface 420, a picture archiving and communications system (PACS) 422, and a server 424.

The processor 412 may be configured to control operations of the preoperative imaging system 130. For example, the processor 412 may be a CPU, a GPU, an APU, a microprocessor, a microcontroller, a DSP, an FPGA, an ASIC, or the like. The processor 412 may be implemented in hardware, firmware, or a combination of hardware and software. The processor 412 may include one or more processors 412 configured to perform the operations described herein. For example, a single processor 412 may be configured to perform all of the operations described herein. Alternatively, multiple processors 412, collectively, may be configured to perform all of the operations described herein, and each of the multiple processors 412 may be configured to perform a subset of the operations descried herein. For example, a first processor 412 may perform a first subset of the operations described herein, a second processor 412 may be configured to perform a second subset of the operations described herein, etc.

The processor 412 may be configured to control the gantry 402, movement of the rotational frame 404, the X-ray source 406, the X-ray detector 408, and movement of the table 410.

The memory 414 may be configured to store information and/or instructions for use by the processor 412. The memory 414 may be a non-transitory computer-readable medium. For example, the memory 414 may be a RAM, a ROM, a flash memory, a magnetic memory, an optical memory, or the like. The memory 414 may be configured to store instructions that, when executed by the processor 412, cause the processor 412 to perform the operations described herein.

The display 416 may be configured to display information. For example, the display 416 may be a monitor, an LED display, a cathode ray tube, a projector display, a touchscreen, tablet computer, mobile phone, or the like.

The user input device 418 may be configured to receive a user input, and provide the user input to the processor 412. For example, the user input device 418 may be a touch screen display, a keyboard, a keypad, a mouse, a button, a switch, a microphone, or the like. Additionally, or alternatively, the user input device 418 may be configured to sense information. For example, the user input device 418 may sense information from an electro-magnetic positioning system, an inertial measurement system, an accelerometer, a gyroscope, an actuator, or the like.

The communication interface 420 may be configured to enable the processor 412 to communicate with other devices, such as via a wired connection, a wireless connection, or a combination of wired and wireless connections. For example, the communication interface 420 may include an Ethernet interface, an optical interface, a coaxial interface, an infrared interface, an RF interface, a USB interface, a Wi-Fi interface, a cellular network interface, or the like. The PACS 422 may be configured to communicate with external systems and/or networks to permit users at various locations to access the medical image. The server 424 may be configured to store one or more models as described herein. For example, the server 424 may be an on-premises server, a cloud server, a virtual machine, or the like.

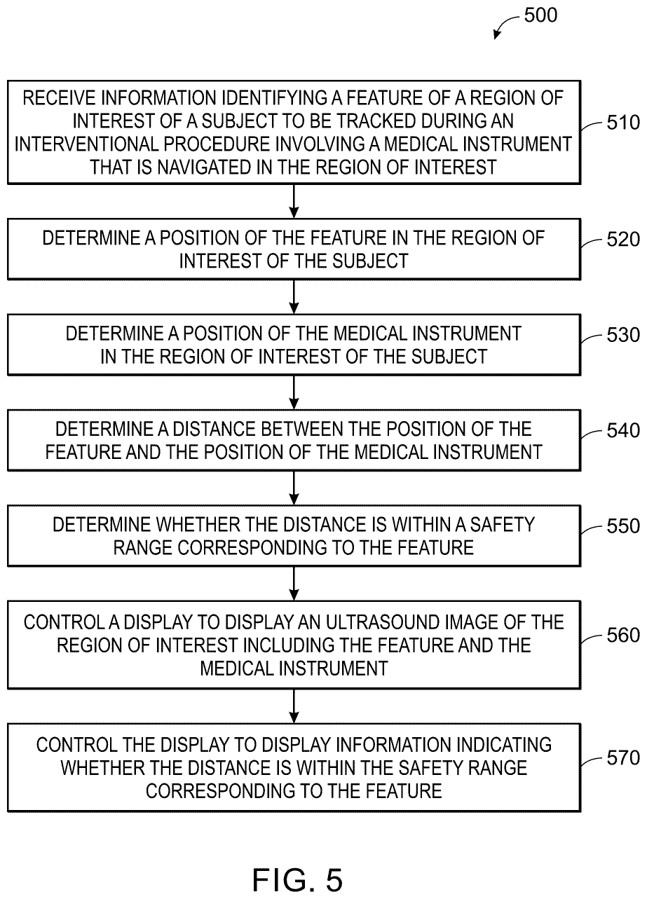

FIG. 5 is a flowchart of an example process for displaying information indicating whether a distance between a position of a feature of a region of interest of a subject and a position of a medical instrument in the region of interest is within a safety range corresponding to the feature.

As shown in FIG. 5, the process 500 may include receiving information identifying a feature of a region of interest of a subject to be tracked during an interventional procedure involving a medical instrument that is navigated in the region of interest (operation 510). For example, the ultrasound system 110 may receive information identifying a feature of a region of interest of a subject to be tracked during an interventional procedure involving the medical instrument 140 that is navigated in the region of interest of the subject.

According to an embodiment, the region of interest may be any region of a subject. For example, the region of interest may be the heart, the brain, the liver, a blood vessel, or the like. The subject may be a patient, an animal, a phantom, or the like. The region of interest may be a region that is associated with an interventional procedure involving the medical instrument 140. For example, the interventional procedure may be a medical procedure involving the medical instrument 140 being navigated in the region of interest.

According to an embodiment, the feature in the region of interest of the subject may be an anatomical feature. For example, in the case where the region of interest is the heart, the feature may be the His bundle, the mitral valve, the tricuspid valve, the sinoatrial node, the atrioventricular node, the left atrial appendage, or the like. Additionally, or alternatively, the feature in the region of interest of the subject may be an implantable device. For example, in the case where the region of interest is the heart, the feature may be a pacemaker, a stent, a defibrillator, a left ventricular assist device, a valve clip, or the like. Additionally, or alternatively, the feature in the region of interest may be a biological substance. For example, in the case where the region of interest is the heart, the feature may be a thrombus, plaque, an inflammation, or the like.

According to an embodiment, the ultrasound system 110 may receive the information identifying the feature in the region of interest based on a user input. For example, a user may interact with a user interface of the ultrasound system 110 to input information identifying the feature. As an example, the ultrasound system 110 may display an ultrasound image of the region of interest, the user may provide a user input that selects a particular feature in the displayed ultrasound image, and the ultrasound system 110 may receive the information identifying the feature based on the user input. As another example, the ultrasound system 110 may display a user interface that includes a list of features, the user may provide a user input that selects a feature from the list of features, and the ultrasound system 110 may receive the information identifying the feature based on the user input.

According to an embodiment, the ultrasound system 110 may receive the information identifying the feature in the region of interest based on subject history information. For example, the subject history information may include a medical record, a medical image, a diagnosis, a procedural history, or the like. The subject history information may identify a feature that is to be tracked during the interventional procedure. For example, the subject history information may identify that the subject includes an implantable device, includes a thrombus, includes a stent, includes an anatomical feature that is susceptible to trauma, or the like.

According to an embodiment, the ultrasound system 110 may receive the information identifying the feature in the region of interest based on detecting the feature in the region of interest. For example, the ultrasound system 110 may acquire an ultrasound image of the region of interest, and detect the feature in the region of interest using the ultrasound image. As another example, the ultrasound system 110 may receive preoperative imaging data from the preoperative imaging system 130, and detect the feature in the region of interest using the preoperative imaging data. In either case, the ultrasound system 110 may detect the feature using a template matching technique (e.g., speckle tracking), an image registration technique, an image segmentation technique, an AI technique, or the like.

According to an embodiment, the ultrasound system 110 may receive information identifying n features of interest in the region of interest to be tracked during the interventional procedure. For example, the ultrasound system 110 may receive information identifying a single feature, two features, five features, etc. In this way, the ultrasound system 110 may identify the particular feature, or features, to be tracked during the interventional procedure.

As shown in FIG. 5, the process 500 may include determining a position of the feature in the region of interest of the subject (operation 520). For example, the ultrasound system 110 may determine the position of the feature in the region of interest of the subject.

According to an embodiment, the ultrasound system 110 may determine the position of the feature in the region of interest based on a user input. For example, the ultrasound system 110 may display a user interface that displays the region of interest, receive a user input that identifies a positon of the feature in the region of interest, and determine the position of the feature in the region of interest based on the user input. A

According to an embodiment, the ultrasound system 110 may determine the position of the feature in the region of interest based on detecting the feature using a template matching technique (e.g., speckle tracking), an image registration technique, an image segmentation technique, an AI technique, or the like. For example, the ultrasound system 110 may segment the feature in the region of interest, and determine the position of the feature in the region of interest based on segmenting the feature in the region of interest. Additionally, or alternatively, the ultrasound system 110 may determine the position of the feature in the region of interest using an AI model. For example, the ultrasound system 110 may input an image of the region of interest into the AI model, and determine the position of the feature in the region of interest based on an output of the AI model that identifies the position of the feature in the region of interest.

According to an embodiment, the ultrasound system 110 may determine the position of the feature in the region of interest based on information received from the preoperative imaging system 130. For example, the preoperative imaging system 130 may provide information that identifies the position of the feature of the region of interest to the ultrasound system 110, and the ultrasound system 110 may determine the position of the feature in the region of interest based on the information received from the preoperative imaging system 130.

According to an embodiment, the ultrasound system 110 may determine the position of the feature in the region of interest based on information received from an external device. For example, the external device may store subject history information that identifies a position of the feature in the region of interest, and provide the subject history information to the ultrasound system 110. In this case, the ultrasound system 110 may determine the position of the feature in the region of interest based on the subject history information received from the external device.

According to an embodiment, the position of the feature in the region of interest may include a set of coordinates in a coordinate system of the ultrasound system 110. For example, the coordinate system of the ultrasound system 110 may be a coordinate system that establishes coordinates of the feature of the region of interest in ultrasound images acquired by the ultrasound system 110.

According to an embodiment, the ultrasound system 110 may determine n positions of n features in the region of interest. For example, the ultrasound system 110 may determine the position of a single feature in the region of interest, may determine the position of two features in the region of interest, may determine the position of five feature in the region of interest, or the like.

According to an embodiment, the ultrasound system 110 may determine the position of the feature in the region of interest before the interventional procedure involving the medical instrument 140. For example, the ultrasound system 110 may acquire ultrasound data of the region of interest before the interventional procedure, and determine the position of the feature in the region of interest using the ultrasound data. Alternatively, the ultrasound system 110 may acquire preoperative imaging data from the preoperative imaging system 130, and determine the position of the feature in the region of interest using the preoperative imaging data.

According to an embodiment, the ultrasound system 110 may determine the position of the feature in the region of interest in substantially real-time during the interventional procedure involving the medical instrument 140. For example, the ultrasound system 110 may determine the position of the feature in the region of interest during the interventional procedure concurrently with the movement of the medical instrument 140 in the region of interest. As used herein, “substantially real-time” may refer to an event occurring within a threshold timeframe of another event, such as within 10 milliseconds, one second, two seconds, etc. In this case, the ultrasound system 110 may acquire ultrasound data of the region of interest during the interventional procedure, and track the positions of the feature in the region of interest using a template matching technique (e.g., speckle tracking), an image registration technique, an AI technique, or the like.

According to an embodiment, the ultrasound system 110 may determine the position of the feature in the region of interest n amount of times. For example, the ultrasound system 110 may determine the position of the feature in the region of interest a single time, and use the determined position during the interventional procedure. Additionally, or alternatively, the ultrasound system 110 may continuously determine the position of the feature in the region of interest at a particular interval, such as every second, every ten seconds, or the like.

According to an embodiment, the ultrasound system 110 may generate a spatial map of the region of interest that identifies the position of the feature in the region of interest, and determine the position of the feature in the region of interest based on the spatial map. The spatial map may identify a position of the feature in the region of interest. For example, the spatial map may include a set of coordinates of the feature in the region of interest in a coordinate system of the ultrasound system 110.

According to an embodiment, the ultrasound system 110 may generate a spatio-temporal map of the region of interest that identifies a set of positions of the feature in the region of interest across a timeframe, and determine positions of the feature in the region of interest based on the spatio-temporal map. The spatio-temporal map of the feature may identify a set of positions of the feature in the region of interest across a timeframe. For example, the spatio-temporal map may include sets of coordinates of the feature in the region of interest in a coordinate system of the ultrasound system 110 across a timeframe. For instance, during the cardiac cycle, a feature of the heart may change positions during contraction and relaxation of the myocardium. In this case, the spatio-temporal map may identify the positions of the feature of the heart during the cardiac cycle.

As further shown in FIG. 5, the process 500 may include determining a position of the medical instrument in the region of interest of the subject (operation 530). For example, the ultrasound system 110 may determine a position of the medical instrument 140 in the region of interest of the subject.

According to an embodiment, the ultrasound system 110 may determine the position of the medical instrument 140 in the region of interest of the subject using ultrasound data acquired by the ultrasound system 110. For example, the ultrasound system 110 may acquire ultrasound data of the region of interest during the interventional procedure, and determine the position of the medical instrument 140 in the region of interest using a template matching technique (e.g., speckle tracking), an image registration technique, an AI technique, or the like. Additionally, or alternatively, the ultrasound system 110 may determine the position of the feature in the region of interest using tracking data acquired by the tracking system 120. For example, the tracking system 120 may acquire tracking data of the medical instrument 140 during the interventional procedure and provide the tracking data to the ultrasound system 110, and the ultrasound system 110 may determine the position of the medical instrument 140 based on the tracking data.

According to an embodiment, the ultrasound system 110 may determine the position of the medical instrument 140 in the region of interest in substantially real-time during the interventional procedure involving the medical instrument 140. For example, the ultrasound system 110 may determine the position of the medical instrument 140 in the region of interest during the interventional procedure concurrently with the movement of the medical instrument 140 in the region of interest.

According to an embodiment, the position of the medical instrument 140 in the region of interest may include a set of coordinates of the medical instrument 140 in the coordinate system of the ultrasound system 110. For example, the coordinate system of the ultrasound system 110 may be the coordinate system that establishes coordinates of the feature of the region of interest in ultrasound images acquired by the ultrasound system 110 and that establishes coordinates of the medical instrument 140 in the region of interest. In this way, the respective coordinates of the feature and the medical instrument 140 can be compared, as described below.

As further shown in FIG. 5, the process 500 may include determining a distance between the position of the feature and the position of the medical instrument (operation 540). For example, the ultrasound system 110 may determine a distance between the position of the feature and the position of the medical instrument 140.

According to an embodiment, the ultrasound system 110 may compare the set of coordinates of the position of the feature and the set of coordinates of the position of the medical instrument 140, and determine the distance based on comparing the set of coordinates of the position of the feature and the set of coordinates of the position of the medical instrument 140.

According to an embodiment, the distance between the position of the feature and the position of the medical instrument 140 may be a distance between an actual real-time position of the feature of the region of interest and an actual real-time position of the medical instrument 140 in the region of interest. For example, the distance may be an actual real-time distance between the feature and the medical instrument 140.

According to an embodiment, the distance between the position of the feature and the position of the medical instrument 140 may be an expected distance between an actual real-time position of the feature of the region of interest and an expected position of the medical instrument 140. For example, the ultrasound system 110 may determine an actual real-time position of the feature, determine an expected position of the medical instrument 140 based on a trajectory of the medical instrument 140, and determine a distance between the actual real-time position of the feature and the expected position of the medical instrument 140.

According to an embodiment, the distance between the position of the feature and the position of the medical instrument 140 may be an expected distance between an expected position of the feature and an actual position of the medical instrument 140. For example, the ultrasound system 110 may determine an expected position of the feature based on the spatio-temporal map, determine an actual real-time position of the medical instrument 140, and determine a distance between the expected position of the feature and the actual real-time position of the medical instrument 140.

According to an embodiment, the distance between the position of the feature and the position of the medical instrument 140 may be an expected distance between an expected position of the feature and an expected position of the medical instrument 140. For example, the ultrasound system 110 may determine an expected position of the feature based on the spatio-temporal map, determine an expected position of the medical instrument 140 based on a trajectory of the medical instrument 140, and determine a distance between the expected position of the feature and the expected position of the medical instrument 140.

In the foregoing situations, the expected position of the feature may be a particular position from the spatio-temporal map. For instance, the expected position may be a position of the feature at maximum contraction of the myocardium, at maximum relaxation of the myocardium, or the like.

In this way, the ultrasound system 110 may determine a distance between a position of the feature in the region of interest and a position of the medical instrument 140 in the region of interest, and determine whether the distance is within a safety range, as described below.

As further shown in FIG. 5, the process 500 may include determining whether the distance is within a safety range corresponding to the feature (operation 550). For example, the ultrasound system 110 may determine whether the distance is within a safety range corresponding to the feature.

According to an embodiment, the safety range may be one or more distances that, if satisfied, result in safety to the subject, result in safe operation of the medical instrument 140, result in safety to the feature in the subject, reduce or prevent harm to the subject, reduce or prevent harm to the feature, or the like. For example, if the distance is within the safety range, then the medical instrument 140 might not harm the subject, might not harm the feature, might not affect the operation of the feature, might not cause health problems, or the like. As another example, if the distance is not within the safety range, then the medical instrument 140 might harm the subject, might harm the feature, might affect the operation of the feature, or the like.

According to an embodiment, the safety range may be a threshold distance. For example, the safety range may be 3 centimeters (cm). In this case, if the distance is less than 3 cm, then the distance is not within the safety range. Alternatively, if the distance is greater than 3 cm, then the distance is within the safety range. It should be understood that the safety range may include any threshold distance, and might depend on the particular feature.

According to an embodiment, the safety range may include a set of tiers. For example, the safety range may include a first tier that is the most safe, a second tier that is less safe than the first tier but safer than a third tier, the third tier that is less safe than both of the first tier and the second tier, etc. According to an embodiment, the safety range may include a single tier including a single safety distance. For example, a first tier of the safety range may be 0-3 cm, a second tier may be 3-6 cm, a third tier may be 6+ cm, or the like.

According to an embodiment, the ultrasound system 110 may receive information identifying the safety range, or may be pre-configured with the information identifying the safety range. The safety range may be established based on clinical guidelines, a user input, or the like.

According to an embodiment, the ultrasound system 110 may determine the safety range based on the particular feature. For example, a first feature may be associated with a first safety range, a second feature may be associated with a third safety range, etc. In other words, the safe, or acceptable, distances between features of the region of interest and the medical instrument 140 might depend on the particular underlying features. As an example, the medical instrument 140 might be able to be positioned very near, or contact, a first feature without causing substantial harm to the subject or the feature, but might not be able to be positioned within a certain distance of a second feature without causing substantial harm to the subject or the feature. It should be understood that different features may include different safety ranges that delineate how closely the medical instrument 140 can be positioned to the features without causing harm.

According to an embodiment, the ultrasound system 110 may compare the distance between the feature in the region of interest and the position of the medical instrument 140 with the safety range, and determine whether the distance is within a safety range corresponding to the feature. For example, the ultrasound system 110 may determine whether the distance is within the safety range. Additionally, or alternatively, the ultrasound system 110 may determine whether the distance is within a particular tier of the safety range. In this way, the ultrasound system 110 may control a display to display information indicating whether the distance is within the safety range, as described below.

As further shown in FIG. 5, the process 500 may include controlling a display to display an ultrasound image of the region of interest including the feature and the medical instrument (operation 560). For example, the ultrasound system 110 may control the display 218 of the ultrasound system 110 to display an ultrasound image of the region of interest including the feature and the medical instrument 140.

According to an embodiment, the ultrasound system 110 may acquire substantially real-time ultrasound images of the region of interest, and display the substantially real-time ultrasound images of the region of interest. The ultrasound images may include the feature and the medical instrument 140. For instance, the ultrasound system 110 may track the feature and the medical instrument 140, and display the ultrasound image to include the tracked feature and the tracked medical instrument 140. In this way, the user can assess the relative positions of the feature and the medical instrument 140, and can assess the distance between the feature and the medical instrument 140.

As further shown in FIG. 5, the process 500 may include controlling the display to display information indicating whether the distance is within the safety range corresponding to the feature (operation 570). For example, the ultrasound system 110 may control the display 218 to display information indicating whether the distance is within the safety range corresponding to the feature.

According to an embodiment, the information indicating whether the distance is within the safety range corresponding to the feature may identify the feature and indicate whether the distance is within the safety range. For example, the information may identify the feature using a label of the feature, a designation of the feature, a highlighting of the feature, a bounding box encircling the feature, or the like. Further, the information indicating whether the distance is within the safety range corresponding to the feature may include a visual indication of whether the distance is within the safety range. For example, the visual indication may be “yes,” “no,” the color green, the color red, “safe,” “unsafe,” or the like. Additionally, or alternatively, the information indicating whether the distance is within the safety range corresponding to the feature may include a visual indication of a discrete value of the distance. For example, the visual indication may be a value for the distance.

According to an embodiment, the information indicating whether the distance is within the safety range corresponding to the feature may include a visual indication of the safety range of the feature. For example, the visual indication of the safety range of the feature may be a plot depicting a lower bound of the safety range and an upper bound of the safety range. The lower bound may be a minimum distance of the safety range, and the upper bound may be a maximum distance of the safety range. Additionally, or alternatively, the visual indication may include a marker that is positioned between the lower bound and the upper bound of the safety range, and that indicates the distance between the feature and the medical instrument 140. In this case, the ultrasound system 110 may update the visual indication in substantially real-time such that the marker moves relative to the lower bound and the upper bound as the distance between the feature and the medical instrument 140 changes as the medical instrument 140 is navigated through the region of interest.

According to an embodiment, the information indicating whether the distance is within the safety range corresponding to the feature may be a warning indicating that the distance is not within the safety range. For example, the ultrasound system 110 may display a warning identifying that the distance between the feature and the medical instrument 140 is unsafe. As described above, the distance may be the actual distance or an expected distance. Accordingly, in the event that the distance is an expected distance, the warning may be a preemptive warning. For example, the warning may indicate that a trajectory of the medical instrument 140 may be unsafe based on the safety range and an expected, or actual, position of the feature.

According to an embodiment, the visual indication of the safety range may be a visual delineation of the metes and bounds of the safety range. For example, the visual indication may be a visual delineation around the feature that identifies the safety range. The visual delineation may include a different image parameter (e.g., color, opacity, hue, intensity, or the like) for regions that are not within the safety range as compared to image parameters of other portions of the ultrasound image that are within the safety range.

According to an embodiment, the information indicating whether the distance is within the safety range corresponding to the feature may be a visual indication including an instruction for the user to move the medical instrument 140 in a particular direction. For example, the visual indication may include an instruction to move the medical instrument 140 in a direction that will result in the distance between the feature and the medical instrument 140 being within the safety range.

According to an embodiment, the ultrasound system 110 may output an audio indication that indicates whether the distance is within the safety range corresponding to the feature. For example, the ultrasound system 110 may output an audio indication based on the occurrence of a particular event. For example, the particular event may be distance between the feature and the medical instrument 140 leaving the safety range and entering the unsafe range. Alternatively, the particular event may be the distance between the feature and the medical instrument 140 leaving a particular tier of the safety range and entering another tier of the safety range. Alternatively, the particular event may be a decrease in the distance between the feature and the medical instrument 140. As another example, the ultrasound system 110 may output an audio indication that identifies a relative distance between the feature and the medical instrument 140. For example, the ultrasound system 110 may display an audio indication having an audio parameter (e.g., level, tone, frequency, or the like) that changes based on the distance between the feature and the medical instrument 140. As a particular example, the ultrasound system 110 may increase a frequency of the audio indications based on a decrease in the distance.

In this way, a user of the medical instrument 140 may have situational awareness as the medical instrument 140 is navigated through the region of interest, and assess whether the medical instrument 140 is within safety ranges of various features of the region of interest. Accordingly, some implementations herein improve subject safety, improve the outcomes of interventional procedures, and increase the efficacy and speed of interventional procedures. Further still, some implementations herein provide an improved user interface for viewing medical images of a region of interest during an interventional procedure.

The number and arrangement of the operations of the process 500 shown in FIG. 5 are provided as an example. In practice, the process 500 may include additional operations, fewer operations, different operations, differently ordered, or differently arranged operations than those shown in FIG. 5.

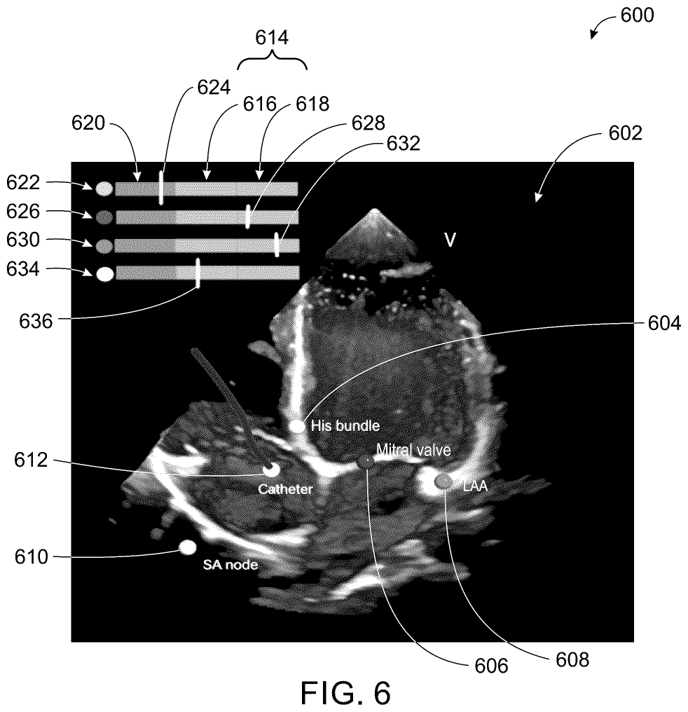

FIG. 6 is a diagram 600 of an ultrasound image displaying features of a region of interest, a medical instrument, safety ranges of the features, and information indicating whether respective positions of the features are within the safety ranges of the features.

As shown in FIG. 6, the ultrasound system 110 may display an ultrasound image 602 of a region of interest (e.g., heart). The ultrasound image 602 may include a visual indication 604 for a first feature (e.g., His bundle), a visual indication 606 of a second feature (e.g., mitral valve), a visual indication 608 for a third feature (e.g., left atrial appendage), a visual indication 610 for a fourth feature (e.g., sinoatrial node), and a visual indication 612 for the medical instrument 140 (e.g., catheter). In this way, the user can visually assess the relative positions of the features with respect to the medical instrument 140 as the user navigates the medical instrument 140 through the region of interest.

As further shown in FIG. 6, the ultrasound system 110 may display a visual indication of a safety range 614 that identifies distances between the respective features and the medical instrument 140 that are considered safe. The safety range 614 may include a first tier 616 that is relatively safe and a second tier 618 that is safer than the first tier 616. Further, the ultrasound system 110 may display an unsafe range 620 that includes distances between the respective features and the medical instrument 140 that are considered unsafe.

As further shown in FIG. 6, the ultrasound system 110 may display a visual indication 622 for the first feature and a visual indication 622 in the form of a marker that identifies a distance between the first feature and the medical instrument 140. Further, the visual indication 622 identifies that the distance between the first feature and the medical instrument 140 is in the unsafe range 620. In this way, a user can assess that the medical instrument 140 is positioned too closely to the first feature and that the medical instrument 140 might cause harm to the first feature and/or the subject.

As further shown in FIG. 6, the ultrasound system 110 may display a visual indication 626 for the second feature and a visual indication 628 in the form of a marker that identifies a distance between the second feature and the medical instrument 140. Further, the visual indication 628 identifies that the distance between the second feature and the medical instrument 140 is in the second tier 618 of the safety range 614. In this way, a user can assess that the medical instrument 140 is positioned safely with respect to the second feature.

As further shown in FIG. 6, the ultrasound system 110 may display a visual indication 630 for the third feature and a visual indication 632 in the form of a marker that identifies a distance between the third feature and the medical instrument 140. Further, the visual indication 632 identifies that the distance between the third feature and the medical instrument 140 is in the second tier 618 of the safety range 614. In this way, a user can assess that the medical instrument 140 is positioned safely with respect to the third feature.

As further shown in FIG. 6, the ultrasound system 110 may display a visual indication 634 for the fourth feature and a visual indication 636 in the form of a marker that identifies a distance between the fourth feature and the medical instrument 140. Further, the visual indication 636 identifies that the distance between the fourth feature and the medical instrument 140 is in the first tier 616 of the safety range 614. In this way, a user can assess that the medical instrument 140 is positioned relatively safely with respect to the fourth feature, but that caution should be exercised to avoid the distance entering the unsafe range 620.

Although FIG. 6 depicts the safety range 614 and the unsafe range 620 as being the same for each of the features, it should be understood that the safety ranges and unsafe ranges may vary based on the particular features.

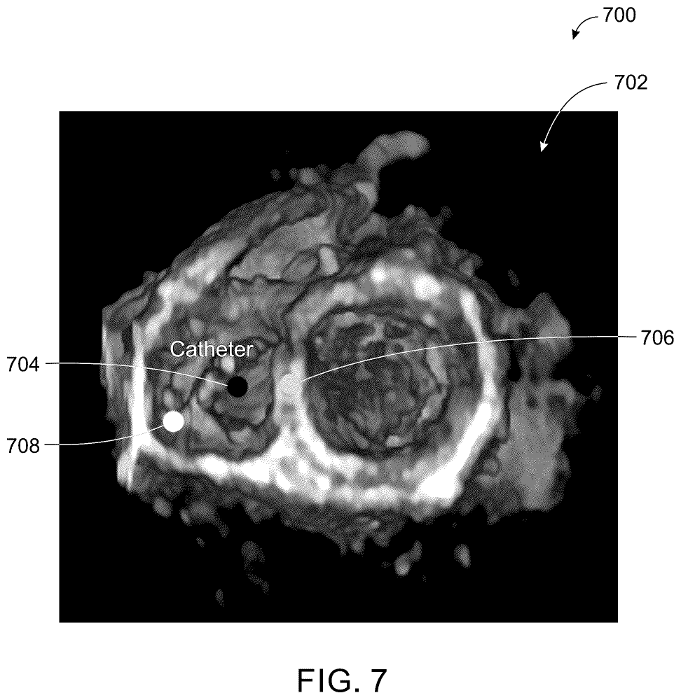

FIG. 7 is a diagram 700 of an ultrasound image displaying features of a region of interest and a medical instrument. As shown in FIG. 7, the ultrasound system 110 may display an ultrasound image 702 including a visual indication of the medical instrument 140, a first feature 706, and a third feature 708. The ultrasound image 702 includes a plane that is perpendicular to the trajectory of the medical instrument 140. In this way, the user can assess the respective positions of the features and the medical instrument 140 from different viewing planes corresponding to the trajectory of the medical instrument 140.

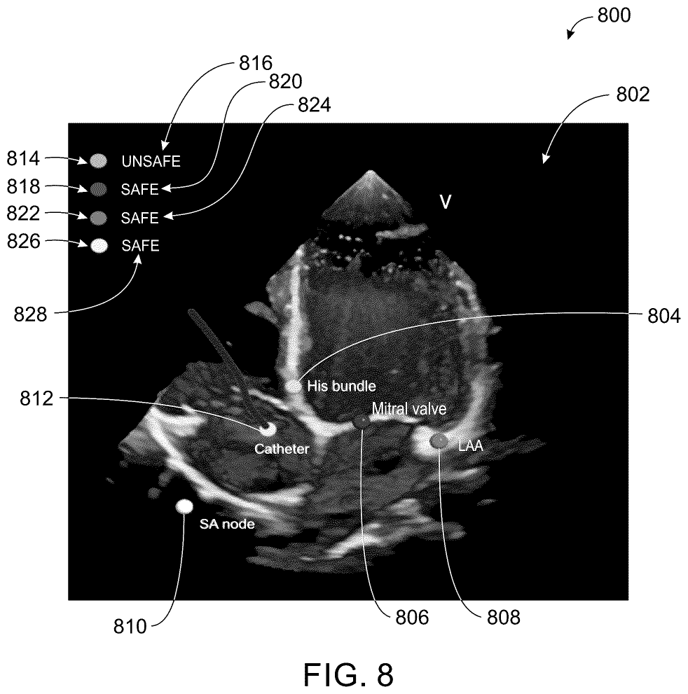

FIG. 8 is a diagram 800 of an ultrasound image displaying features of a region of interest, a medical instrument, and information indicating whether respective positions of the features are within the safety ranges of the features.

As shown in FIG. 8, the ultrasound system 110 may display an ultrasound image 802 of a region of interest (e.g., heart). The ultrasound image 802 may include a visual indication 804 for a first feature (e.g., His bundle), a visual indication 806 of a second feature (e.g., mitral valve), a visual indication 808 for a third feature (e.g., left atrial appendage), a visual indication 810 for a fourth feature (e.g., sinoatrial node), and a visual indication 812 for the medical instrument 140 (e.g., catheter). In this way, the user can visually assess the relative positions of the features with respect to the medical instrument 140 as the user navigates the medical instrument 140 through the region of interest.

As further shown in FIG. 8, the ultrasound system 110 may display a visual indication 814 for the first feature and a visual indication 816 identifying that the medical instrument 140 is not within the safety range corresponding to the first feature. As further shown in FIG. 8, the ultrasound system 110 may display a visual indication 818 for the second feature and a visual indication 820 identifying that the medical instrument 140 is within the safety range corresponding to the second feature. As further shown in FIG. 8, the ultrasound system 110 may display a visual indication 822 for the third feature and a visual indication 824 identifying that the medical instrument 140 is within the safety range corresponding to the third feature. As further shown in FIG. 8, the ultrasound system 110 may display a visual indication 826 for the fourth feature and a visual indication 826 identifying that the medical instrument 140 is within the safety range corresponding to the fourth feature. In this way, the user can assess whether the medical instrument 140 is positioned safely, or unsafely, with respect to the features.

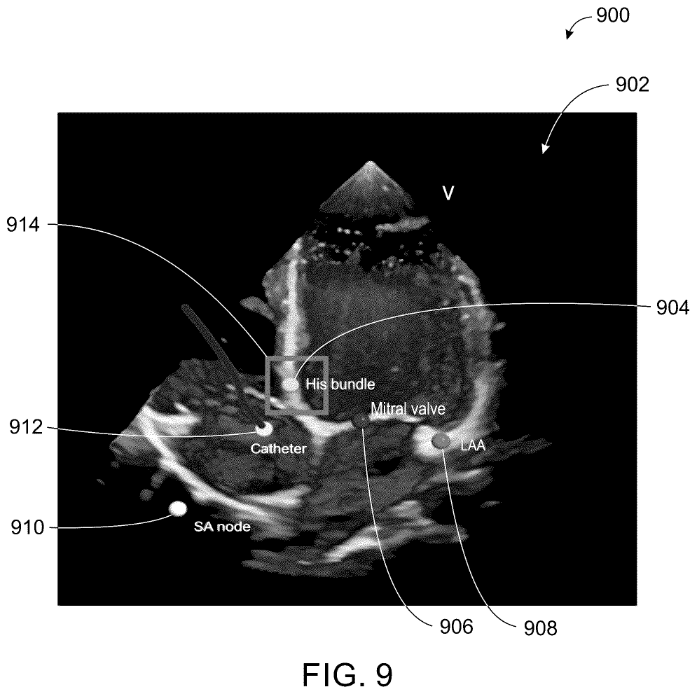

FIG. 9 is a diagram 900 of an ultrasound image displaying features of a region of interest, a medical instrument, and information indicating whether respective positions of the features are within the safety ranges of the features.

As shown in FIG. 9, the ultrasound system 110 may display an ultrasound image 902 of a region of interest (e.g., heart). The ultrasound image 902 may include a visual indication 904 for a first feature (e.g., His bundle), a visual indication 906 of a second feature (e.g., mitral valve), a visual indication 908 for a third feature (e.g., left atrial appendage), a visual indication 910 for a fourth feature (e.g., sinoatrial node), and a visual indication 912 for the medical instrument 140 (e.g., catheter). In this way, the user can visually assess the relative positions of the features with respect to the medical instrument 140 as the user navigates the medical instrument 140 through the region of interest. As further shown in FIG. 9, the ultrasound system 110 may display a visual indication 914 identifying that the medical instrument 140 is not within the safety range corresponding to the first feature. In this way, the user can assess that the medical instrument 140 is positioned safely, or unsafely, with respect to the features.

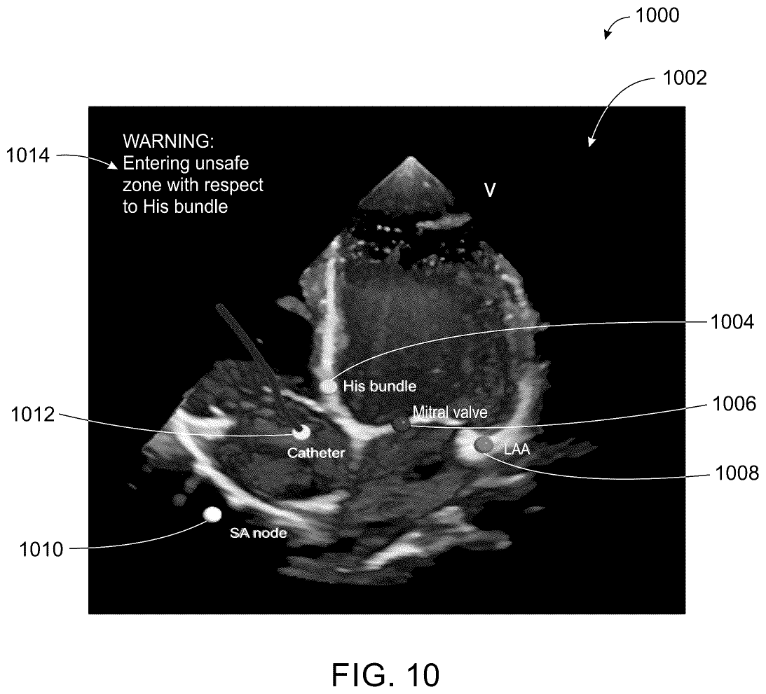

FIG. 10 is a diagram 1000 of an ultrasound image displaying features of a region of interest, a medical instrument, and information indicating whether respective positions of the features are within the safety ranges of the features.

As shown in FIG. 10, the ultrasound system 110 may display an ultrasound image 1002 of a region of interest (e.g., heart). The ultrasound image 1002 may include a visual indication 1004 for a first feature (e.g., His bundle), a visual indication 1006 of a second feature (e.g., mitral valve), a visual indication 1008 for a third feature (e.g., left atrial appendage), a visual indication 1010 for a fourth feature (e.g., sinoatrial node), and a visual indication 1012 for the medical instrument 140 (e.g., catheter). In this way, the user can visually assess the relative positions of the features with respect to the medical instrument 140 as the user navigates the medical instrument 140 through the region of interest. As further shown in FIG. 10, the ultrasound system 110 may display a visual indication 1014 identifying that the medical instrument 140 is on a trajectory that will result in the medical instrument 140 being positioned outside of the safety range corresponding to the first feature. In this way, the user can assess that the medical instrument 140 is positioned safely, or unsafely, with respect to the features.

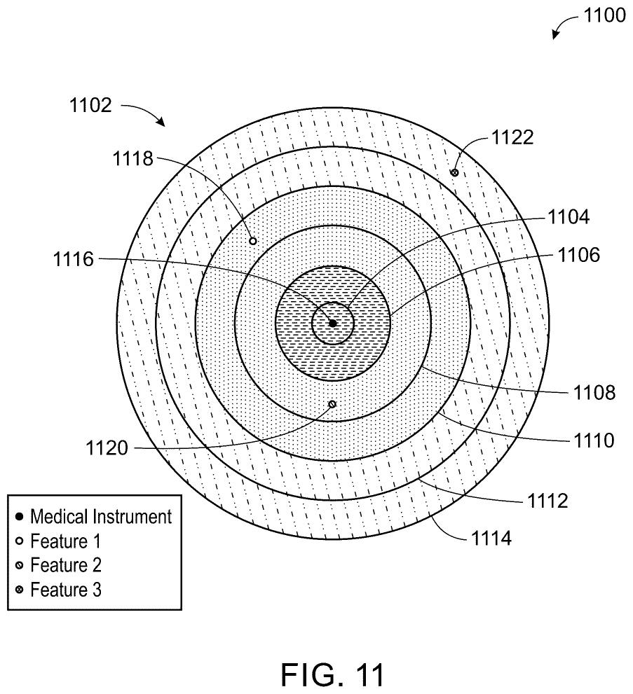

FIG. 11 is a diagram 1100 of a map displaying features of a region of interest, a medical instrument, and information indicating whether respective positions of the features are within the safety ranges of the features.

As shown in FIG. 11, the ultrasound system 110 may display a map 1102 including a set of tiers of an unsafe range of the medical instrument 140 relative to a set of features, and a set of tiers of a safety range of the medical instrument 140 relative to the set of features. For example, the map 1102 may include a first tier 1104 of an unsafe range of the medical instrument 140 relative to the set of features, and a second tier 1106 of the unsafe range of the medical instrument 140 relative to the set of features. In this case, the first tier 1104 may be more unsafe as compared to the second tier 1106. As further shown, the map 1102 may include a first tier 1108 of a safety range of the medical instrument 140 relative to the set of features, a second tier 1110 of the medical instrument 140 relative to the set of features, a third tier 1112 of the safety range of the medical instrument 140 relative to the set of features, and a fourth tier 1114 of the medical instrument 140 relative to the set of features. The first tier 1104, the second tier 1106, the first tier 1108, the second tier 1110, the third tier 1112, and the fourth tier 1114 may be displayed with different image parameters (e.g., color, opacity, hue, intensity, or the like).

The ultrasound system 110 may display a visual indication 1116 of the medical instrument 140 on the map 1102. The visual indication 1116 may be fixed at the center of the map 1102. The ultrasound system 110 may display a visual indication 1118 of a first feature of the region of interest. The visual indication 1118 may include a position on the map 1102 that identifies a distance of the first feature relative to the medical instrument 140, that identifies a position of the first feature relative to the medical instrument 140, and that identifies whether the distance is within a safety range of the first feature. Here, the visual indication 1118 is in the second tier 1110 of the safety range, which indicates that the distance is relatively safe. The ultrasound system 110 may display a visual indication 1120 of a second feature of the region of interest. The visual indication 1120 may include a position on the map 1102 that identifies a distance of the second feature relative to the medical instrument 140, that identifies a position of the second feature relative to the medical instrument 140, and that identifies whether the distance is within a safety range of the second feature. Here, the visual indication 1120 is in the first tier 1108 of the safety range, which indicates that caution should be exercised. The ultrasound system 110 may display a visual indication 1120 of a third feature of the region of interest. The visual indication 1122 may include a position on the map 1102 that identifies a distance of the third feature relative to the medical instrument 140, that identifies a position of the third feature relative to the medical instrument 140, and that identifies whether the distance is within a safety range of the third feature. Here, the visual indication 1122 is in the fourth tier 1114 of the safety range, which indicates that the distance is relatively safe.

The ultrasound system 110 may update the respective positions of the visual indication 1118, the visual indication 1120, and the visual indication 1122 as the medical instrument 140 is navigated throughout the region of interest. In this way, the user can visually assess the relative positions of the features with respect to the medical instrument 140 as the user navigates the medical instrument 140 through the region of interest.

The ultrasound system 110 may overlay the map 1102 on an ultrasound image, such as the ultrasound image 602, the ultrasound image 702, the ultrasound image 802, the ultrasound image 902, and/or the ultrasound image 1002.

Although the implementations herein are described as being performed using ultrasound data and ultrasound images, it should be understood that the implementations herein are applicable to other imaging modalities. Further, although the implementations herein are described as being performed in conjunction with cardiac procedures, it should be understood that the implementations herein are applicable to any other type of interventional procedure involving any anatomical region of a subject.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A system comprising:
an ultrasound probe configured to acquire ultrasound data of a region of interest of a subject;
a display configured to display an ultrasound image corresponding to the ultrasound data;
a memory configured to store instructions; and
one or more processors configured to execute the instructions to:
receive information identifying a feature in the region of interest of the subject to be tracked during an interventional procedure involving a medical instrument that is navigated through the region of interest;
determine a position of the feature in the region of interest of the subject;
determine a position of the medical instrument in the region of interest of the subject during the interventional procedure;
determine a distance between the position of the feature and the position of the medical instrument;
determine whether the distance is within a safety range corresponding to the feature;
control the display to display the ultrasound image of the region of interest including the feature and the medical instrument; and
control the display to display information indicating whether the distance between the position of the feature and the position of the medical instrument is within the safety range corresponding to the feature.

2. The system of claim 1, wherein the information indicating whether the distance between the position of the feature and the position of the medical instrument is within the safety range corresponding to the feature includes a marker that indicates the distance between the position of the feature and the position of the medical instrument and that moves relative to a lower bound of the safety range.

3. The system of claim 1, wherein the information indicating whether the distance between the position of the feature and the position of the medical instrument is within the safety range corresponding to the feature includes a visual indication of the safety range.

4. The system of claim 1, wherein the information indicating whether the distance between the position of the feature and the position of the medical instrument is within the safety range corresponding to the feature includes a warning indicating that the distance is not within the safety range.

5. The system of claim 1, wherein the one or more processors are further configured to:
generate a spatio-temporal map of the region of interest that identifies a set of positions of the feature in the region of interest across a timeframe,
wherein the determining the distance between the position of the feature and the position of the medical instrument comprises determining the distance between the position of the feature and the position of the medical instrument using the spatio-temporal map.

6. The system of claim 1, wherein the one or more processors are further configured to:
control the display to display information indicating whether respective distances between respective positions of multiple features and the position of the medical instrument are within respective safety ranges corresponding to the multiple features.

7. The system of claim 1, wherein the feature includes an anatomical feature or an implantable device.

8. A method comprising:
receiving information identifying a feature in a region of interest of a subject to be tracked during an interventional procedure involving a medical instrument that is navigated through the region of interest;
determining a position of the feature in the region of interest of the subject;
determining a position of the medical instrument in the region of interest of the subject during the interventional procedure;
determining a distance between the position of the feature and the position of the medical instrument;
determining whether the distance is within a safety range corresponding to the feature;
controlling a display to display an ultrasound image of the region of interest corresponding to the ultrasound data including the feature and the medical instrument; and
controlling the display to display information indicating whether the distance between the position of the feature and the position of the medical instrument is within the safety range corresponding to the feature.

9. The method of claim 8, wherein the information indicating whether the distance between the position of the feature and the position of the medical instrument is within the safety range corresponding to the feature includes a marker that indicates the distance between the position of the feature and the position of the medical instrument and that moves relative to a lower bound of the safety range.

10. The method of claim 8, wherein the information indicating whether the distance between the position of the feature and the position of the medical instrument is within the safety range corresponding to the feature includes a visual indication of the safety range.

11. The method of claim 9, wherein the information indicating whether the distance between the position of the feature and the position of the medical instrument is within the safety range corresponding to the feature includes a warning indicating that the distance is not within the safety range.

12. The method of claim 9, further comprising:
generating a spatio-temporal map of the region of interest that identifies a set of positions of the feature in the region of interest across a timeframe,
wherein the determining the distance between the position of the feature and the position of the medical instrument comprises determining the distance between the position of the feature and the position of the medical instrument using the spatio-temporal map.

13. The method of claim 9, further comprising:
controlling the display to display information indicating whether respective distances between respective positions of multiple features and the position of the medical instrument are within respective safety ranges corresponding to the multiple features.

14. The method of claim 9, wherein the feature includes an anatomical feature or an implantable device.

15. A non-transitory computer-readable medium storing instructions that, when executed by one or more processors, cause the one or more processors to:
receive information identifying a feature in a region of interest of a subject to be tracked during an interventional procedure involving a medical instrument that is navigated through the region of interest;
determine a position of the feature in the region of interest of the subject;
determine a position of the medical instrument in the region of interest of the subject during the interventional procedure;
determine a distance between the position of the feature and the position of the medical instrument;
determine whether the distance is within a safety range corresponding to the feature;
control a display to display an ultrasound image of the region of interest corresponding to the ultrasound data including the feature and the medical instrument; and
control the display to display information indicating whether the distance between the position of the feature and the position of the medical instrument is within the safety range corresponding to the feature.

16. The non-transitory computer-readable medium of claim 15, wherein the information indicating whether the distance between the position of the feature and the position of the medical instrument is within the safety range corresponding to the feature includes a marker that indicates the distance between the position of the feature and the position of the medical instrument and that moves relative to a lower bound of the safety range.

17. The non-transitory computer-readable medium of claim 15, wherein the information indicating whether the distance between the position of the feature and the position of the medical instrument is within the safety range corresponding to the feature includes a visual indication of the safety range.

18. The non-transitory computer-readable medium of claim 15, wherein the information indicating whether the distance between the position of the feature and the position of the medical instrument is within the safety range corresponding to the feature includes a warning indicating that the distance is not within the safety range.

19. The non-transitory computer-readable medium of claim 15, wherein the one or more processors are further configured to:
control the display to display information indicating whether respective distances between respective positions of multiple features and the position of the medical instrument are within respective safety ranges corresponding to the multiple features.

20. The non-transitory computer-readable medium of claim 15, wherein the feature includes an anatomical feature or an implantable device.
